(19) United States
(12) Patent Application Publication
(10) Pub. No.: US 2012/0101427 A1
(43) Pub. Date: Apr. 26, 2012

(54) NOVEL PHOTOSENSITIZER FORMULATIONS FOR ORAL ADMINISTRATION

(76) Inventors: Gerard Farmer, Muenchenstein (CH); Gerhard Wieland, Jena (DE); Dietrich Scheglmann, Jena (DE); Arno Wiehe, Berlin (DE); Susanna Gräfe, Jena (DE); Nikolay E. Nufantiev, Moscow (RU); Volker Albrecht, Nuthetal (DE); Wolfgang Neuberger, Dubai (AE)

(21) Appl. No.: 13/266,056

(22) PCT Filed: Apr. 28, 2009
(86) PCT No.: PCT/US2010/032780

§ 371 Date: Jan. 6, 2012

Related U.S. Application Data
(60) Provisional application No. 61/173,477, filed on Apr. 28, 2009.

(51) Int. Cl. A61M 37/00; A61K 31/498; C07D 487/22; C07D 241/46; A61K 9/127; A61K 38/02; A61K 31/7056; A61K 31/706; A61K 31/79; A61P 35/00; A61P 17/00; A61P 9/00; A61P 19/02; A61P 31/04; A61P 33/02; A61P 31/12; A61P 31/00; A61K 31/409; B82Y 5/00

(52) U.S. Cl. 604/20; 514/410; 514/250; 540/145; 544/347; 540/122; 540/472; 424/450; 514/1.1; 514/23; 424/78.24; 977/773; 977/906; 977/788

(57) ABSTRACT

The present invention provides novel drug formulations for oral administration for diverse medical applications including anticancer, antimetastatic, antibacterial, antifungal, antiprotozoic, antiviral, antiprionic and PDT treatments for diagnostic and therapeutic purposes. In a preferred embodiment the oral drug formulation comprises a photosensitizer and suitable excipients and may be administered in multiple doses over an extended period of time with exposure to activating radiation occurring generally between individual doses or in a light-independent manner. In another preferred embodiment PDT methods for treating hyperplasia and neoplasia, for localizing hyperplasic and neoplasic tissues and pathogen bacteria by fluorescence, for treating infections caused by pathogen bacteria in complex body fluids and for fat reduction, skin disorders and vascular diseases are provided.

### DOMESTIC PRIORITY UNDER 35 USC 119(E)

This application claims the benefit and priority of U.S. Provisional Application Ser. No. 61/173,477 filed Apr. 28, 2009, entitled “NOVEL PHOTOSENSITIZER FORMULATIONS FOR ORAL ADMINISTRATION” by Gerard Farmer et al., which is incorporated by reference herein.

### BACKGROUND OF THE INVENTION

1. Field of the invention

The present invention relates to the field of novel formulations for oral administration. More particularly, the present invention relates to novel oral photosensitizer formulations for anticancer, antimetastatic, antibacterial, antifungal, antiprotozoic, antiviral, antiprionic and PDT treatments.

2. Prior Art Disclosure Statement

Depending on the subcellular target, photosensitizers may produce different damaging effects. Some photosensitizers induce cell self-destruction due to their ability to generate active proteases, such as caspase while other photosensitizers are likely to induce apoptosis because they localize or are produced in mitochondria. Hydrophobic photosensitizers present an increased affinity to neoplasic tissues. Aggregates as well as hydrophilic photosensitizers are likely to be taken up by pinocytosis and/or endocytosis and are localized in lysosomes and endosomes. When activated, vesicles become permeable and photosensitizers and hydrolytic enzymes are released into the cytosol. Sensitizing dyes in the cytosol can damage tubulin leading to accumulation of cells in mitosis followed by cell death. Thus, many applications depending on the photosensitizers' sub-cellular targets may be developed.

Although most photosensitizers are usually formulated to be delivered by invasive routes such as intravenous or subcutaneous injections, the development of effective photosensitizer formulations for oral administration would be highly advantageous. This can be achieved by increasing the oral absorption of the photosensitizer through the use of formulations that protect the macromolecule and/or enhance its uptake through the gastrointestinal tract.

In an attempt to provide stable compounds, in U.S. Pat. No. 6,376,483 Robinson primarily discloses bacteriochlorins and bacteriopurpurins compounds or a pharmaceutically acceptable salt, solvate, prodrug or metabolite. Instead of focusing on the synthesis of a novel compound, U.S. Pat. No. 7,364,754 by Prasad et al. provides a drug-carrier system in which ceramic nanoparticles entrap the photosensitive drug 2-devinyl-2(1-hexyloxyethyl) pyropheophorbide. To inhibit cell proliferation and angiogenesis in a light-independent manner, pheophorbide derivative compounds are administered orally as disclosed in WO Patent Publication N^{o} 2008/002460 A2 by Brooks et al.

By administering thiazine blue orally or parenterally, US Patent Publication N^{o} 2006/0264423 A1 and WO Patent Publication N^{o} 2006/127482 A1 by Wood et al. disclose a method for treating hepatitis virus in a patient and a method for decreasing or preventing reactivation of some viruses in a patient. To prevent or decrease reactivation of some viruses, the individual is a patient that is or will be undergoing chemotherapy and is or will be immunosuppresed. Thiazine blue may be exposed to non-ionizing radiation to enhance the anti-viral activity of the dye. Other pharmaceutical presentation for peroral administration of phenothiazine dyes is disclosed in WO Patent Publication N^{o} 2007/144048 comprising an active agent-carrier complex. The carrier is an ion exchanger which eliminates or strongly reduces the tissue- and textile-staining properties of methylene blue as only releases very small amounts of it under physiological conditions of the oral and pharyngeal cavity.

With oral administration it is possible to overcome the difficulties associated with intravenous or subcutaneous routes which require the intervention of a physician or other health care professional, providing long-term control of diseases with minimal patient discomfort. Enteric route has all the advantages of a non-invasive procedure. Unfortunately, it may present some uptake difficulties associated with the size, solubility and stability of the active substance to be delivered such as poor absorption of macromolecules, gastrointestinal destruction of labile molecules and impediment to go through gastrointestinal biological barriers. Thus, to ensure an appropriate active substance bioavailability, the absorption of bioactive agents in an unaltered form and avoidance of massive spleen or liver accumulation should be guaranteed. This can be done by incorporation of the active substance into oral drug delivery systems or novel designed drug formulations.

Thus, there is a need for new photosensitizer formulations with better technical and physicochemical properties than prior art compounds for oral administration. Present invention fulfills the needs of prior art formulations providing enhanced bioavailability and stable photosensitizer formulations for oral administration easy to manufacture and handle. Moreover, the present invention provides photosensitizer formulations for oral administration to improve effectiveness of anticancer, antimetastatic, antibacterial, antifungal, antiprotozoic, antiviral, antiprionic and PDT treatments for a wide variety of applications.

### OBJECTIVES AND BRIEF SUMMARY OF THE INVENTION

It is an objective of the present invention to provide novel photosensitizer formulations to be administered by the oral route for light-independent treatments or therapies activated by light, such as photodynamic therapies.

It is another objective of the present invention to provide novel photosensitizer formulations for oral administration and anticancer and PDT treatment methods to treat hyperplasic and neoplastic cells and tissues.

It is another objective of the present invention to provide novel oral photosensitizer formulations and antimetastatic and PDT treatment methods to treat and prevent neoplastic cells and tissues and metastasis by initial tumor destruction and for long term-tumor control.

It is another objective of the present invention to provide novel oral photosensitizer formulations and antibacterial, antifungal, antiprotozoic, antiviral, antiprionic and PDT treatment methods to treat or prevent infections caused by pathogens in the body and in complex body fluids.

It is another objective of the present invention to provide novel oral photosensitizer formulations and PDT methods to localize hyperplastic and neoplastic tissues and pathogenic bacteria by fluorescence.

It is yet another objective of the present invention to provide novel photosensitizer formulations for oral administration and PDT methods for fat reduction, skin disorders hair treatments and vascular diseases.

Briefly stated the present invention provides novel drug formulations for oral administration to treat diverse medical applications including anticancer, antimetastatic, antibacterial, antifungal, antiprotozoic, antiviral, antiprionic and PDT treatments for diagnostic and therapeutic purposes. In a preferred embodiment, the oral drug formulation comprises a photosensitizer and suitable excipients and may be administered in multiple doses over an extended period of time with exposure to activating radiation occurring generally between individual doses or in a light-independent manner. In another preferred embodiment, PDT methods for treating hyperplasia and neoplasia, for localizing hyperplasic and neoplasic tissues and pathogen bacteria by fluorescence, for treating infections caused by pathogen bacteria in complex body fluids and for fat reduction, skin disorders and vascular diseases are provided.

The above, and other objects, features and advantages of the present invention will become apparent from the following description read in conjunction with the accompanying drawings.

### BRIEF DESCRIPTION OF FIGURES

FIG. 1 illustrates examples of some preferred photosensitizer structure families, including tetrapyrroles derivatives and phenazine dyes.

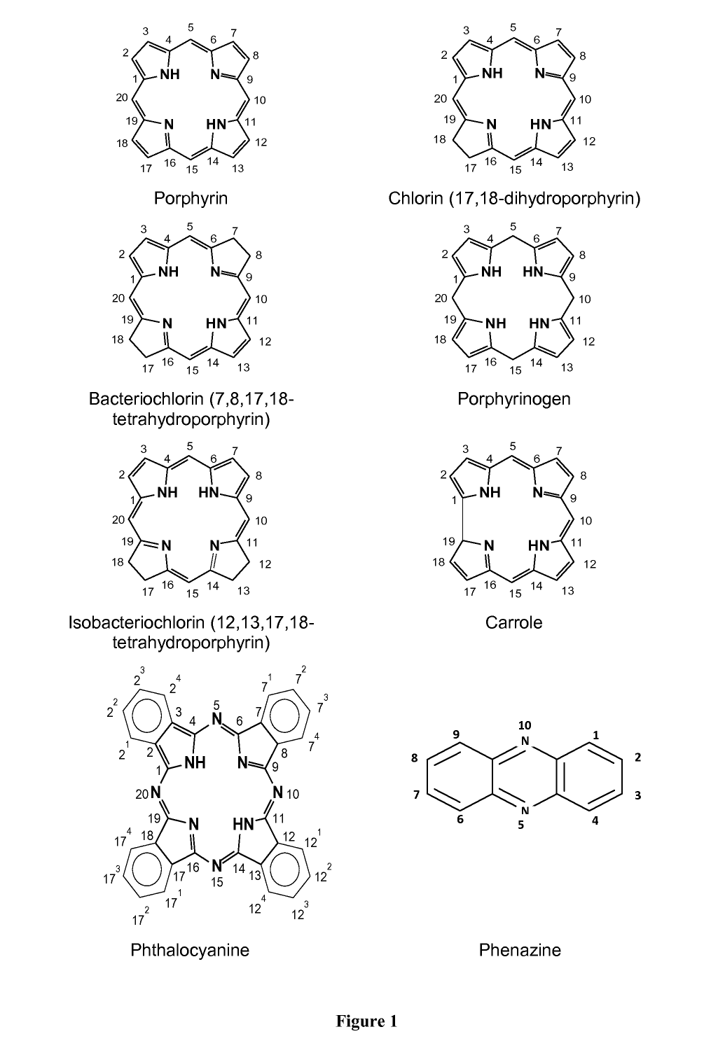

### DETAILED DESCRIPTION OF PREFERRED EMBODIMENTS

In order to provide non-invasive and effective anticancer, antimetastatic, antibacterial, antiprotozoic, antiviral, antiprionic and PDT treatments, novel photosensitizer formulations for oral administration are provided. Present invention provides stable compounds with improved bioavailability of the photosensitizers and enhanced bio-distribution in target tissues. Moreover, present invention offers optimized treatment regimes to minimize light sensitivity and to maximize the potential of the drug while at the same time limiting necroses and causing sequential killing of the target tissue such as cancer dysplasia or other unwanted tissue, even fat, while maximizing beneficial body support reactions such as immune system support.

Among the existing routes of administration, the present invention photosensitizer formulations may be administered orally or involving any part of the gastrointestinal tract such as mouth, pharynx, esophagus, stomach, small intestine (duodenum, jejunum, ileum), large intestine (cecum, colon, rectum) and anus.

Active substances may be administered with assimilable edible carriers, inert diluents or incorporated directly with food. Pharmaceutical dosage form includes but it is not limited to hard or soft shell gelatin capsule, tablet, pill, powder, solution, suspension, elixir, syrup, wafer, gel, buccal or sublingual tablet, thin film, suppository and enema.

Different pharmaceutical excipients for pharmaceutical dosage forms may be employed depending on the particular medical application. Pharmaceutical excipients may be used to modulate the solubility and bioavailability of the photosensitizer, increase its stability, help to maintain preferred polymorphic forms and conformations, maintain pH and/or osmolarity of liquid formulations, modulate immunogenic responses of the host and act as emulsifying agent, antioxidant, aerosol propellants, tablet binders, tablet disintegration agents. Preferred pharmaceutical excipient includes, but it is not limited to binders/fillers, coating agents, disintegration agents, lubricants and sweeteners compatible with the photosensitizer used.

In order to enhance the bioavailability of the oral photosensitizer formulation of present invention and allow its passage through the different gastrointestinal tract barriers, the oral photosensitizer formulation further comprises means for blocking the active efflux mechanism without harming the patient. The active efflux mechanism is performed by multiple-drug efflux pumps (MDR) present on the surface of epithelial cells of the duodenum and the small intestine which operate as active transporters by an active efflux mechanism expelling substances considered toxic outside the cell. The most abundant and most active of these MDR pumps are p-glycoprotein (MDR1, ABCB1), BCRP (Breast Cancer Resistance Protein (ABCG2)) and MRP-2. Some photosensitizers have shown poor bioavailability because they act as substrate for MDR efflux pumps. Therefore, the oral photosensitizer formulation of present invention also comprises MDR-pump-blocking agents including but not limited to Vitamin-E-TPGS, Cremophor EL/RH40, Solutol HS, Tween 20, Tween 80, Labrasol, Peceol, PEGs, Polysorbate 80, Brij 30 and Pluronic P85.

Additionally, novel photosensitizer formulations for oral administration of present invention may be produced without any formulation additives or with other drug delivery systems known in the art such as combinations with liposome forming components, vectored and non-vectored proteins, organic and inorganic nanoparticles, nano- and micro-emulsions, nanocrystals, individual solvents or appropriate solvent mixtures, components like lactose, PVP and others.

In preferred embodiments photosensitizer formulations for oral administration may be used in a light-independent manner or may be activated by an electromagnetic radiation source including coherent and incoherent radiation sources such as laser radiation source, light emitting diodes source, lamp radiation source (incandescent, xenon arc and metal halide lamps) and/or sunlight or other radiation sources from the environment. Depending on the medical application electromagnetic radiation may be delivered transdermally, inside a body cavity or lumen or interstitially by optical fibers with or without diffuser tips.

In a most preferred embodiment a photosensitizer formulation for oral administration for treating hyperplasia and neoplasia is provided. Photosensitizers are preferably tetrapyrroles and their derivates and phenazine dyes and their derivatives, selected from the group consisting of, but not limited to porphyrins, chlorins, bacteriochlorins, pheophorbide, bacteriopheophorbide, corroles and phthalocyanines.

An embodiment of a PDT method for the treatment of hyperplasia and neoplasia comprises the steps of: 1) selecting appropriate amount of photosensitizer in the oral dosage form, preferably tetrapyrrole and their derivatives; 2) administering orally a single or multiple pharmaceutical dosage forms; 3) allowing a period of time for drug absorption at proper sites of the GI tract and preferential accumulation of the photosensitizer at the hyperproliferative tissue; 4) delivering light radiation of one or more wavelengths single or multiple times to activate the photosensitizer accumulated at the treatment area; 5) if needed repeating the treatment more times.

In another medical application, PDT induces a tumor-specific immune reaction that could be mediated by immune cells such as macrophages and/or dendritic cells which serve as antigen presenting cells. In contrast to most other cancer therapies, PDT can induce immunity even against less immunogenic tumors providing a systemic immune response effect. Thus, multiple photosensitizer dosage forms are orally administered for long-term tumor control, preventing the recurrence of PDT-treated tumors. Administration of multiple dosage forms of present invention can stimulate immune cells to be sensitized by the tumor and then they would be able to eliminate small foci of viable cancer cells that have escaped other PDT mediated antitumor effect. Moreover, long-term tumor control by stimulating immune response may prevent development of metastasis and/or development of other primary tumors. An embodiment of PDT method for long-term control of neoplasia comprises the steps of: 1) selecting therapeutically effective amount of photosensitizer in the oral dosage form, preferably tetrapyrrole and their derivatives; 2) administering orally a pharmaceutical dosage form; 3) after administration allowing a period of time for drug absorption at proper sites of the GI tract and preferential accumulation of the photosensitizer at the hyperproliferative tissue; 4) delivering light radiation of one or more wavelengths to activate the photosensitizer for stimulating tumor-immune response; 5) allowing a period of time for photosensitizer replenishment at the treating hyperproliferative tissue by other body tissues; 6) delivering light radiation of one or more wavelengths to activate replenished photosensitizer for stimulation of host tumor-immune response; 7) after a suitable period of time repeating steps 1-6 multiple times for long-term anti-neoplastic control.

Main advantages of PDT long-term anti-neoplastic control via oral route of administration comprise the administration of lower effective doses of photosensitizer compared to prior art formulations, thus limiting unintended necrosis and reducing extended skin photo-sensitivity. Additionally, this approach increases immune system support by stimulating host tumor-immune response and increases effectiveness by using for each photosensitizer administration multiple irradiation steps, after allowing photosensitizer replenishment at treated sites.

In a most preferred embodiment a photosensitizer formulation for oral administration for treating infections caused by pathogen bacteria in complex body fluids such as whole blood, blood products, saliva and others is provided. Photosensitizers are preferably phenazine dyes and/or their derivatives, selected from the group consisting of, but not limited to methylene blue, safranin, etc. An embodiment of a PDT method of treatment of pathogen bacteria in complex body fluids comprises the steps of: 1) selecting appropriate amount of photosensitizer in the oral dosage form, preferably safranin O; 2) administering orally a single or multiple pharmaceutical dosage forms; 3) allowing a period of time for drug absorption at proper sites of the GI tract and preferential accumulation of the photosensitizer at the infected tissue; 4) delivering light radiation of one or more wavelengths to activate the photosensitizer accumulated at the treatment area, preferably in an intermittent manner; 5) allowing a period of time for photosensitizer replenishment at the treating infected tissue by other body tissues; 6) delivering light radiation of one or more wavelengths to activate replenished photosensitizer; 7) after a suitable period of time repeating steps 1-6 multiple times for long-term antibacterial control. The present method provides long-term antibacterial control by sequentially killing or inactivating infective agents such as bacteria.

In another embodiment, a photosensitizer formulation to be administered by the oral route for antiprionic treatments is provided. Prions are infectious pathogens that cause neurodegenerative diseases involving the modification of the prion protein (PrP). Apparently, the accumulation in the central nervous system of the abnormal protease-resistant form of PrP results in prion disease. Bovine spongiform encephalopathy, scrapie of sheep, and Creutzfeldt-Jakob disease of humans are among the most notable transmissible spongiform encephalopathies or prion diseases. Certain porphyrins and phthalocyanines may act as inhibitors of the abnormal protease-resistant PrP accumulation and may also inhibit the conversion to abnormal protease-resistant PrP without apparent cytotoxic effect.

In another preferred embodiment a photosensitizer formulation for oral administration for localizing hyperplasic or neoplasic tissues and bacteria by fluorescence is provided. A PDT method for localizing hyperplasic or neoplasic tissues and bacteria by fluorescence via the oral administration of appropriate amount of photosensitizer in a proper oral dosage form allows visualization of the target material with the aid of a fluorescence microscope or any other appropriate means. In another embodiment an advantageous combined method includes diagnosis and treatment of hyperplasic or neoplasic tissue in the same treatment. As the photosensitizer preferentially accumulates in the hyperproliferative tissue the area is first illuminated with a light radiation absorbed by the photosensitizer but with shallow penetration depth, allowing accurate visualization of the area to be treated. Then, the selected areas to be treated should be illuminated with a light radiation absorbed by the photosensitizer but with deeper penetration depth to activate the photosensitizer and destroy the hyperproliferative tissue.

When excess body fat is accumulated in a human subject health may be negatively affected. Body Mass Index (BMI) is a number calculated from a person's weight and height. It provides a reliable indicator of body fat accumulation for most people. For adults, standard weight status categories are associated with BMI. A BMI less than 18.5 kg/m² indicates underweight, a BMI between 25-29.9 kg/m² indicates overweight and a BMI of 30 kg/m² or higher indicates obesity. Excess body fat may affect a subject physically, physiologically and psychologically and is associated with aesthetical disorders and various diseases such as cardiovascular diseases, diabetes mellitus type II, obstructive sleep apnea and others. In another embodiment of the present invention, photosensitizer formulations for oral administration are used in PDT treatments for fat reduction by destroying undesired fat cells in the body. After identifying selective areas to be treated and administering appropriate amount of photosensitizer in oral dosage form, the photosensitizer is progressively accumulated in adipose cells in the subcutaneous layer. After a dwell time light radiation of one or more wavelengths is delivered to activate the photosensitizer accumulated at treatment areas to reduce or eliminate adipose tissue. Light radiation delivery may be performed multiple times allowing replenishment of photosensitizer from other body tissues to the treating adipose areas between light irradiations. The photodynamic treatment can be performed multiple times until desired fat elimination or reduction is achieved. Light radiation may be laser or LED radiation delivered with the aid of a lamp or an optical fiber. Irradiation may be delivered transdermally with the aid of an appropriate handpiece or interstitially with the aid an optical fiber with a diffuser tip.

PDT has been also used for neoplastic and non-neoplastic dermatological disorders such as basal cell carcinoma, actinic keratoses, viral warts, acne and others. In another embodiment, photosensitizer formulations for oral administration are used in PDT treatments for skin disorders in which the photosensitizer accumulated and activated by light radiation at the treatment area produces cytotoxic effects on neoplastic and non-neoplastic unhealthy skin cells.

In another embodiment, photosensitizer formulations for oral administration of the present invention are used for hair PDT treatments. Different PDT applications for hair treatments may be obtained depending on the amount of photosensitizer administered, the interval between light and drug administration (DLI: drug-light interval) and irradiation parameters such as power intensity per treated area, energy delivered per treated area, continuous or pulsed irradiation and others. In one embodiment, PDT may be utilized for removal of unwanted hair in human subjects or animals. The extension of area to be treated and properties of hair to be removed would determine whether multiple or single drug and/or light administration are needed and the precise photosensitizer amount therapeutically effective. Present method allows undesired hair elimination by inactivating or destroying the hair follicles or destroying the tissue feeding the hair follicles.

Nevertheless, if different drug and illumination parameter's settings are used PDT treatment can stimulate hair growth by inducing, reviving, renewing, replacing or activating hair growth. Preferably, PDT stimulates hair growth of hairs produced by follicles with sebaceous glands which are found on the scalp, beard, arm and pubic areas. Thus, hair loss such as androgenetic alopecia, chemotherapy and drug-induced alopecia and alopecia areata can be treated with appropriate PDT settings.

In another embodiment, photosensitizer formulations for oral administration are used in PDT treatments for vascular disorders. PDT may be utilized for treating varicose and spider veins by administering appropriate amount of photosensitizer in oral dosage form. Once the photosensitizer is preferentially accumulated or adhered to unhealthy vein wall vessels and/or achieves appropriate blood concentration, pulsed or continuous light radiation of one or more wavelengths is delivered endoluminally or transdermally to activate the photosensitizer. By a photodynamic process the vein wall or endothelium is damaged and/or irritated leading to immediate or progressive vein closure by irreversible evolution into fibrotic tissue. Preferably photosensitizers are tetrapyrroles and their derivatives, irradiated with a laser source of wavelengths in the range of 400 to 800nm.

A variety of joint disorders affects many people in all age groups, some of which are of chronic nature. The main symptom of such disorders is acute pain. Due to decreased movement because of pain, regional muscles may gradually atrophy and ligaments may become more lax, leading over time to deformity and disability. One of the most common joint disorders is rheumatoid arthritis, a disease affecting million of people in United States. To reduce pain, conservative cares such as weight control, rest, regular exercise or mechanical support devices may be helpful. But if pain increases medical treatments are required, including non steroidal anti-inflammatory drugs, local injections of glucocorticoid or hyaluronan and in severe cases, joint replacement surgery. In another embodiment, the present invention provides an alternative non-invasive prophylactic and treatment method to enhance cartilage regeneration and provide people suffering from this disease an improved quality of life.

The present invention is further illustrated by the following examples, but is not limited thereby.

### EXAMPLE 1

### Capsule Formulation

Depending on the desired drug delivery profile, absorption site and active substance properties, tablet formulations may include but are not limited to the following: Capsule 1) Hypromellose, Ethylcellulose, Lactose monohydrate, Magnesium stearate; Capsule shell: Titanium dioxide (E 171), Yellow iron oxide (E 172), Red iron oxide (E 172), Gelatin. Printing ink (Opacode S-1-15083): Shellac, Lecithin (soya), Simethicone, Red iron oxide (E 172), Hydroxypropyl Cellulose; Capsule 2) Hypromellose, Hydroxypropyl methylcellulose acetate succinate, Sucrose, Sugar spheres, Talc, Titanium dioxide (E171), Triethyl citrate; Capsule shell: Gelatin, Sodium Lauryl Sulfate, Titanium Dioxide (E171), Indigo Carmine (E132), Yellow Iron Oxide (E172), Edible White Ink; Capsule 3) Lactose monohydrate, Magnesium Stearate, Povidone, Silica, colloidal anhydrous/Colloidal silicon dioxide, Polysorbate 20; Capsule shell: Gelatin, Titanium dioxide (E171), Iron oxide, red (E172). In all cases, the active substance is the photosensitizer. The amount of active substance would be determined depending on the desired therapeutic application in order to achieve the optimal therapeutic effect.

### EXAMPLE 2

### Tablet Formulation

In case larger amounts of active substance needs to be delivered, capsule formulations might be too big compared to compressed tablet, thus the following tablet formulations may be produced: Tablet 1) Tablet core: Maize starch, Pregelatinised starch, Sodium starch glycollate, Povidone, Glycerol dibehenate, Magnesium stearate; Film coat: Hypromellose, Glycerol triacetate, Talc, Titanium dioxide (E171), Iron oxide yellow (E172), Iron oxide red (E172), Ethylcellulose; Tablet 2) Lactose monohydrate, Powdered cellulose, Pregelatinised maize starch, Maize starch, Colloidal anhydrous silica, Magnesium stearate; Tablet 3) Maize starch, Microcrystalline cellulose, Hydroxypropyl cellulose, Magnesium stearate, Indigo carmine aluminium lake; Tablet 4) Tablet core: Lactose monohydrate, Microcrystalline Cellulose, Crospovidone, Silica, colloidal anhydrous/Colloidal silicon dioxide, Magnesium stearate; Film coat: Polyvinyl alcohol—part hydrolised Titanium dioxide, Talc, Lecithin, Xanthan gum. In all cases, the active substance is the photosensitizer. The amount of active substance would be determined depending on the desired therapeutic application in order to achieve the optimal therapeutic effect.

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

What is claimed is:

1. An oral formulation of a hydrophobic photosensitizer.

2. The oral drug formulation according to claim 1 comprising a photosensitizer and suitable excipients, wherein said photosensitizers can be of any structure other than anthraquinone deivatives or aliphatic amines.

3. The oral drug formulation according to claim 1 comprising a photosensitizer and suitable excipients, wherein said photosensitizers are tetrapyrroles and their derivatives or phenazine dyes and their derivatives.

4. The oral drug formulation according to claim 2 or 3, wherein said suitable excipients include multiple-drug efflux pump-blocking agents, wherein said multiple-drug efflux pump-blocking agents are selected from the group consisting of Vitamin-E-TPGS, Cremophor EL/RH40, Solutol HS, Tween 20, Tween 80, Labrasol, Peceol, PEGs, Polysorbate 80, Brij 30, Pluronic P85 and combinations of them.

5. The oral drug formulation according to claim 2 or 3, wherein said suitable excipients are selected from the group consisting of solvent, solubilizing agents, emulsifiers, adjuvants, wetting agents, suspending agents, crystallization inhibitors, preservatives, pH buffering agents, sweeteners, flavouring, odor masking agents, fillers, binders, coating agents, disintegration agents, lubricants, glidants, buffering agents, coloring agents, solution retarding agents, absorption accelerator agents and combinations of them.

6. The oral drug formulation according to claim 2 or 3, having an oral dosage form selected from the group consisting of solutions, suspensions, emulsions, syrups, elixirs, pastes, gels, tablets, capsules, soft capsules, hard capsules, gelatin capsules, pills, powders, granules, premixes, suppository, enema and combinations of them.

7. The oral drug formulation according to claim 2 or 3, wherein said photosensitizer is itself adsorbed on, included in or covalently attached to drug delivery systems which are selected from the group consisting of liposome forming components, vectored and non-vectored proteins, organic and inorganic nanoparticles, nano- and micro-emulsions, nanocrystals, individual solvents, appropriate solvent mixtures, lactose, polyvinylpyrrolidone (PVP) and combinations of them.

8. An orally administered drug formulation of a photo-drug comprising a photosensitizer, which is not significantly degraded by stomach digestive material including acids or enzymes, nor accumulates in liver or kidneys, and an inert ingredient, where necessary, wherein said formulation is useful for treating hyperplasic diseases and in antimicrobial therapy.

9. The oral drug formulation according to claim 2 or 3, wherein dosage is set for multiple dosing over an extended time with exposure to activating radiation occurring generally between individual doses.

10. The oral drug formulation according to claim 2 or 3, wherein dosage is set for multiple dosing over an extended time for radiation-independent treatments.

11. The oral drug formulation according to claim 9, wherein dosage is set for multiple dosing over an extended time with exposure to activating radiation occurring generally between individual doses.

12. The oral drug formulation according to claim 9, wherein dosage is set for multiple dosing over an extended time for radiation-independent treatments.

13. A method of treating tumors, dysplasias or other medical or cosmetic conditions by administering a photosensitizer orally, allowing time for it to accumulate in target tissue, followed by applying suitable energy to activate said photosensitizer in the target tissue.

14. A method for treating medical and cosmetic conditions such as fat removal, dermatological disorders, hair removal, hair growth, vascular disorders, joint disorders by administering a hydrophobic photosensitizer orally, allowing time for it to accumulate in target tissue, followed by applying suitable energy to activate said photosensitizer in the target tissue.

15. A method of treating tumors, dysplasias, other medical/cosmetic conditions comprising the steps of:
a) selecting appropriate amount of hydrophobic photosensitizer in oral dosage form;
b) administering orally in single or multiple pharmaceutical dosage forms;
c) allowing a period of time for drug absorption at proper sites of GI tract and preferential accumulation of the photosensitizer at hyperproliferative tissue (treatment area);
d) delivering light radiation of one or more wavelengths, single or multiple times to activate the photosensitizer accumulated at the treatment area;
e) if needed repeating the treatment more than once.

16. A method of treating tumors and dysplasias for long-term tumor control which induces a tumor-specific immune reaction comprising the steps of:
a) selecting therapeutically effective amount of hydrophobic photosensitizer in oral dosage form;
b) administering orally a pharmaceutical dosage form;
c) after administration allowing a period of time for drug absorption at proper sites of the GI tract and preferential accumulation of the photosensitizer at hyperproliferative tissue;
d) delivering light radiation of one or more wavelengths to activate the photosensitizer for stimulating tumor-immune response;
e) allowing a period of time for photosensitizer replenishment at the treating hyperproliferative tissue by other body tissues;
f) delivering light radiation of one or more wavelengths to activate replenished photosensitizer for stimulation of host tumor-immune response;
g) after a suitable period of time repeating steps a-f multiple times for long-term anti-neoplastic control.

17. A method of treating bacterial, protozoic, viral and prionic infections or other medical conditions comprising the steps of:
a) selecting appropriate amount of hydrophobic photosensitizer in oral dosage form;
b) administering orally in single or multiple pharmaceutical dosage forms;
c) allowing a period of time for drug absorption at proper sites of the GI tract and preferential accumulation of the photosensitizer at the infected tissue;
d) delivering light radiation of one or more wavelengths to activate the photosensitizer accumulated at the treatment area;
e) allowing a period of time for photosensitizer replenishment at the treating infected tissue by other body tissues;
f) delivering light radiation of one or more wavelengths to activate replenished photosensitizer;
g) after a suitable period of time repeating steps a-f multiple times for long-term antibacterial control.

19. The method of treating infections and other medical conditions according to claim 18, wherein the selected hydrophobic photosensitizer is Safranin O.

20. The method of treating infections and other medical conditions according to claim 18, wherein said delivering power is done in an intermittent manner and endoluminally.

21. The method of treating tumors, dysplasias, other medical/cosmetic conditions according to claim 16 or 17 wherein in said step a) selecting is from the group of tetrapyrroles and their derivatives.
